SEQUENCE LISTING (68 sequences)

SEQ ID NO: 1 - protein, 414 aa, Artificial Sequence
  Phytase Chimera PTRec 77
SDTAPAGFQLERVVILSRHGVRAPTKMTQTMRDVTPHQWPEWPVKLGYITPRGEHLISLMGGFYRERFQQQGLLPKDNCPTPDAVYVWADVDQRTRLTGQAFLDGIAPGCGLKVHYQADLEKVDPLFHPVEAGVCQLDSTQTHRAIEAQLGAPLSELSQRYAKPFAQMSEILNFTASPYCKSLQQQGKSCDFATFAANEVKVNQQGTKVSLSGPLALSSTLGEVFLLQEAQGMPQVAWGRITGEKEWRDLLSLHNAQFDLLQRTPYLAKHNGTPLLQTIAHALGSNITSRPLPDISPDNKILFIAGHDTNIANISGMLGMTWTLPGQPDNTPPGGALVFERWVDNAGKPYVSVNMVYQTLAQLHDQAPLTLQHPAGSVRLNIPGCSDQTPDGYCPLSTFSRLVSHSVEPACQLP

SEQ ID NO: 2 - protein, 415 aa, Artificial Sequence
  Phytase Chimera PTRec 74
APVAAPVTGYTLERVVILSRHGVRSPTKQTELMNNVTPDKWPQWPVPAGYLTPRGAQLVKLMGGFYGDYFRNQGLLPAGCPADGTLYAQADIDQRTRLTGQAFLDGIAPGCGLKVHYQADLEKVDPLFHPVEAGVCQLDSTQTHRAIEAQLGAPLSELSQRYAKPFAQMSEILNFTASPYCKSLQQQGKSCDFATFAANEVKVNQQGTKVSLSGPLALSSTLGEVFLLQEAQGMPQVAWGRITGEKEWRDLLSLHNAQFDLLQRTPYLAKHNGTPLLQTIAHALGSNITSRPLPDISPDNKILFIAGHDTNIANISGMLGMTWTLPGQPDNTPPGGALVFERWVDNAGKPYVSVNMVYQTLAQLHDQAPLTLQHPAGSVRLNIPGCSDQTPDGYCPLSTFSRLVSHSVEPACQLP

SEQ ID NO: 3 - DNA, 1245 bp, Artificial Sequence
  phy PTRec 77
tctgatactgctccagctggtttccagttggagagagttgttatcttgtccagacacggtgttagagctccaactaagatgactcagactatgagagatgttactccacaccaatggccagagtggccagttaagttgggttacattactccaagaggtgagcacttgatttctttgatgggtggtttctacagagagagattccagcagcaaggtttgttgccaaaggacaactgtccaactccagacgctgtttacgtttgggctgatgttgaccagagaactagattgactggtcaggctttcttggacggtattgctccaggttgtggtttgaaggttcactaccaagctgacttggagaaggttgaccctttgttccatccagttgaggctggtgtttgtcagttggactctactcaaactcacagagctatcgaggctcaattgggtgctccattgtctgagttgtcccagagatacgctaagccattcgctcagatgtccgagatcttgaacttcactgcttccccatactgtaaatccttgcagcaacagggaaagtcctgtgactttgctactttcgctgctaacgaggttaaggttaaccagcaaggtactaaggtttccttgtccggtccattggctttgtcctctactttgggtgaggttttcttgttgcaagaggctcagggtatgccacaagttgcttggggtagaatcactggtgagaaagagtggagagacttgttgtccttgcacaacgctcagttcgacttgttgcagagaactccatacttggctaagcacaacggtacacctttgttgcagactatcgctcacgctttgggttccaacattacttccagaccattgccagacatctccccagacaacaagattttgttcattgctggtcacgacacaaacattgctaacatctccggtatgttgggtatgacttggactttgccaggtcaacctgataacactccaccaggtggtgctttggttttcgaaagatgggttgacaacgctggtaagccatacgtttccgttaacatggtttaccagactttggctcagttgcacgaccaagctccattgacattgcaacaccctgctggttccgttagattgaacattccaggttgttccgaccagactcctgatggttactgtccattgtccactttctctagattggtttcccactccgttgagccagcttgtcagttgccataa

SEQ ID NO: 4 - DNA, 1248 bp, Artificial Sequence
  phy PTRec 74
gctccagttgctgctccagttactggttacactttggagagagttgttatcttgtccagacacggtgttagatccccaactaagcagactgagttgatgaacaacgttactccagacaagtggccacaatggccagttccagctggttacttgactccaagaggtgctcagttggttaagttgatgggtggtttctacggtgactacttcagaaaccagggtttgttgcctgctggttgtccagctgatggtactttgtacgctcaagctgacatcgaccagagaactagattgactggtcaggctttcttggacggtattgctccaggttgtggtttgaaggttcactaccaagctgacttggagaaggttgaccctttgttccatccagttgaggctggtgtttgtcagttggactctactcaaactcacagagctatcgaggctcaattgggtgctccattgtctgagttgtcccagagatacgctaagccattcgctcagatgtccgagatcttgaacttcactgcttccccatactgtaaatccttgcagcaacagggaaagtcctgtgactttgctactttcgctgctaacgaggttaaggttaaccagcaaggtactaaggtttccttgtccggtccattggctttgtcctctactttgggtgaggttttcttgttgcaagaggctcagggtatgccacaagttgcttggggtagaatcactggtgagaaagagtggagagacttgttgtccttgcacaacgctcagttcgacttgttgcagagaactccatacttggctaagcacaacggtacacctttgttgcagactatcgctcacgctttgggttccaacattacttccagaccattgccagacatctccccagacaacaagattttgttcattgctggtcacgacacaaacattgctaacatctccggtatgttgggtatgacttggactttgccaggtcaacctgataacactccaccaggtggtgctttggttttcgaaagatgggttgacaacgctggtaagccatacgtttccgttaacatggtttaccagactttggctcagttgcacgaccaagctccattgacattgcaacaccctgctggttccgttagattgaacattccaggttgttccgaccagactcctgatggttactgtccattgtccactttctctagattggtttcccactccgttgagccagcttgtcagttgccataa

SEQ ID NO: 5 - protein, 411 aa, Artificial Sequence
  Citrobacter braakii phytase
EEQNGMKLERVVIVSRHGVRAPTKFTPIMKDVTPDQWPQWDVPLGWLTPRGGELVSELGQYQRLWFTSKGLLNNQTCPSPGQVAVIADTDQRTRKTGEAFLAGLAPKCQIQVHYQKDEEKNDPLFNPVKMGKCSFNTLKVKNAILERAGGNIELYTQRYQSSFRTLENVLNFSQSETCKTTEKSTKCTLPEALPSEFKVTPDNVSLPGAWSLSSTLTEVFLLQEAQGMPQVAWGRITGEKEWRDLLSLHNAQFDLLQRTPYLARSRATPLLDMIDTALLTNGTTENRYGIKLPVSLLFIAGHDTNLANLSGALDLKWSLPGQPDNTPPGGELVFEKWKRTSDNTDWVQVSFVYQTLRDMRDIQPLSLEKPAGKVDLKLIACEEKNSQGMCSLKSFSRLIKEIRVPECAVTE

SEQ ID NO: 6 - protein, 413 aa, Artificial Sequence
  Hafnia alvei phytase
SDTAPAGFQLERVVILSRHGVRAPTKMTQTMRDVTPHQWPEWPVKLGYITPRGEHLISLMGGFYRERFQQQGLLPKDNCPTPDAVYVWADVDQRTRKTGEAFLAGLAPQCDLAIHHQQNTQQADPLFHPVKAGICSMDKSQVHAAVEKQAGTPIETLNQRYQASLALMSSVLDFPKSPYCQQHNIGKLCDFSQAMPSRLAINDDGNKVALEGAVGLASTLAEVFLLEHAQGMPKVAWGNIHTEQQWNSLLKLHNAQFDLMSRTPYLAKHNGTPLLQTIAHALGSNITSRPLPDISPDNKILFIAGHDTNIANISGMLGMTWTLPGQPDNTPPGGALVFERWVDNAGKPYVSVNMVYQTLAQLHDQAPLTLQHPAGSVRLNIPGCSDQTPDGYCPLSTFSRLVSHSVEPACQLP

SEQ ID NO: 7 - protein, 417 aa, Artificial Sequence
  Yersinia mollaretii phytase M1
APVAAPVTGYTLERVVILSRHGVRSPTKQTELMNNVTPDKWPQWPVPAGYLTPRGAQLVKLMGGFYGDYFRNQGLLPAGCPADGTLYAQADIDQRTRLTGQAFLDGIAPGCGLKVHYQADLEKVDPLFHPVEAGVCQLDSTQTHRAIEAQLGAPLSELSQRYAKPFAQMSEILNFTASPYCKSLQQQGKSCDFATFAANEVKVNQQGTKVSLSGPLALSSTLGEVFLLQNSQGMPDVAWHRLSGAENWVSLLSLHNAQFDLMAKTPYLARHKGTPLLQQIMTALVLQRKGQGQTLPLSEQTKLLFLGGHDTNIANIGGMLGANWQLPQQPDNTPPGGGLVFELWQNPDNHQQYVAVKMFYQTMDQLRNSEKLDLKSHPAGIVPIEIEGCENIGTDKLCQLDTFQKRVAQVIEPACHI

SEQ ID NO: 8 - protein, 413 aa, Artificial Sequence
  Phytase Chimera DJ 15
SDTAPAGFQLERVVILSRHGVRAPTKMTQTMRDVTPHQWPEWPVKLGYITPRGEHLISLMGGFYRERFQQQGLLPKDNCPTPDAVYVWADVDQRTRKTGEAFLAGLAPQCDLAIHHQQNTQQADPLFHPVKAGICSMDKSQVHAAVEKQAGTPIETLNQRYQASLALMSSVLDFPKSPYCQQHNIGKLCDFSQAMPSRLAINDDGNKVALEGAVGLASTLAEVFLLQEAQGMPQVAWGRITGEKEWRDLLSLHNAQFDLLQRTPYLAKHNGTPLLQTIAHALGSNITSRPLPDISPDNKILFIAGHDTNIANISGMLGMTWTLPGQPDNTPPGGALVFERWVDNAGKPYVSVNMVYQTLAQLHDQAPLTLQHPAGSVRLNIPGCSDQTPDGYCPLSTFSRLVSHSVEPACQLP

SEQ ID NO: 9 - protein, 417 aa, Artificial Sequence
  Phytase Chimera DJ 2
APVAAPVTGYTLERVVILSRHGVRSPTKQTELMNNVTPDKWPQWPVPAGYLTPRGAQLVKLMGGFYGDYFRNQGLLPAGCPADGTLYAQADIDQRTRLTGQAFLDGIAPGCGLKVHYQADLEKVDPLFHPVEAGVCQLDSTQTHRAIEAQLGAPLSELSQRYAKPFAQMSEILNFTASPYCKSLQQQGKSCDFATFAANEVKVNQQGTKVSLSGPLALSSTLGEVFLLQEAQGMPQVAWGRITGEKEWRDLLSLHNAQFDLLQRTPYLARHKGTPLLQQIMTALVLQRKGQGQTLPLSEQTKLLFLGGHDTNIANIGGMLGANWQLPQQPDNTPPGGGLVFELWQNPDNHQQYVAVKMFYQTMDQLRNSEKLDLKSHPAGIVPIEIEGCENIGTDKLCQLDTFQKRVAQVIEPACHI

SEQ ID NO: 10 - protein, 415 aa, Artificial Sequence
  Phytase Chimera DJ 19
SDTAPAGFQLERVVILSRHGVRAPTKMTQTMRDVTPHQWPEWPVKLGYITPRGEHLISLMGGFYRERFQQQGLLPKDNCPTPDAVYVWADVDQRTRLTGQAFLDGIAPGCGLKVHYQADLEKVDPLFHPVEAGVCQLDSTQTHRAIEAQLGAPLSELSQRYAKPFAQMSEILNFTASPYCKSLQQQGKSCDFATFAANEVKVNQQGTKVSLSGPLALSSTLGEVFLLQEAQGMPQVAWGRITGEKEWRDLLSLHNAQFDLLQRTPYLAKHNGTPLLQTIAHALGSNITSRPLPDISPDNKILFIAGHDTNIANIGGMLGANWQLPQQPDNTPPGGGLVFELWQNPDNHQQYVAVKMFYQTMDQLRNSEKLDLKSHPAGIVPIEIEGCENIGTDKLCQLDTFQKRVAQVIEPACHI

SEQ ID NO: 11 - protein, 416 aa, Artificial Sequence
  Phytase Chimera DJ 24
APVAAPVTGYTLERVVILSRHGVRSPTKQTELMNNVTPDKWPQWPVPAGYLTPRGAQLVKLMGGFYGDYFRNQGLLPAGCPADGTLYAQADIDQRTRLTGQAFLDGIAPGCGLKVHYQADLEKVDPLFHPVEAGVCQLDSTQTHRAIEAQLGAPLSELSQRYAKPFAQMSEILNFTASPYCKSLQQQGKSCDFATFAANEVKVNQQGTKVSLSGPLALSSTLGEVFLLQEAQGMPQVAWGRITGEKEWRDLLSLHNAQFDLLQRTPYLAKHNGTPLLQTIAHALGSNITSRPLPDISPDNKILFIAGHDTNIANIGGMLGANWQLPQQPDNTPPGGGLVFELWQNPDNHQQYVAVKMFYQTMDQLRNSEKLDLKSHPAGIVPIEIEGCENIGTDKLCQLDTFQKRVAQVIEPACHI

SEQ ID NO: 12 - DNA, 19 bp, Artificial Sequence
  Primer
gcggccgctcaagaggatg

SEQ ID NO: 13 - DNA, 24 bp, Artificial Sequence
  Primer
ccttagcagcaatggaagcgatag

SEQ ID NO: 14 - DNA, 24 bp, Artificial Sequence
  Primer
attgctgctaaggaagagggtgtc

SEQ ID NO: 15 - DNA, 20 bp, Artificial Sequence
  Primer
cttgagcggccgcttacaag

SEQ ID NO: 16 - DNA, 24 bp, Artificial Sequence
  Primer
tctagttctctggtcaacgtcagc

SEQ ID NO: 17 - DNA, 28 bp, Artificial Sequence
  Primer
gaccagagaactagaaagactggtgaag

SEQ ID NO: 18 - DNA, 29 bp, Artificial Sequence
  Primer
caacaagaaaacctcagtcaacatggaag

SEQ ID NO: 19 - DNA, 22 bp, Artificial Sequence
  Primer
gaggttttcttgttgcagcagg

SEQ ID NO: 20 - DNA, 22 bp, Artificial Sequence
  Primer
agccaagtatggagttctctgc

SEQ ID NO: 21 - DNA, 30 bp, Artificial Sequence
  Primer
tccatacttggctagatccagagctactcc

SEQ ID NO: 22 - DNA, 22 bp, Artificial Sequence
  Primer
gtttgtgtcgtgaccagcaatg

SEQ ID NO: 23 - DNA, 26 bp, Artificial Sequence
  Primer
tcacgacacaaacttggctaacttgg

SEQ ID NO: 24 - DNA, 23 bp, Artificial Sequence
  Primer
tctagttctctggtctggggaag

SEQ ID NO: 25 - DNA, 28 bp, Artificial Sequence
  Primer
gaccagagaactagagctactgctcaag

SEQ ID NO: 26 - DNA, 27 bp, Artificial Sequence
  Primer
caacaagaaaacctcggacatgttagc

SEQ ID NO: 27 - DNA, 33 bp, Artificial Sequence
  Primer
gaggttttcttgttggagtacactgagaacttg

SEQ ID NO: 28 - DNA, 28 bp, Artificial Sequence
  Primer
agccaagtatggaacgtcgttagtgaag

SEQ ID NO: 29 - DNA, 27 bp, Artificial Sequence
  Primer
tccatacttggctcaaagaggtggttc

SEQ ID NO: 30 - DNA, 25 bp, Artificial Sequence
  Primer
gtttgtgtcgtgagcaacgtacaac

SEQ ID NO: 31 - DNA, 27 bp, Artificial Sequence
  Primer
tcacgacacaaacattgcttacttgag

SEQ ID NO: 32 - DNA, 21 bp, Artificial Sequence
  Primer
cttgagcggccgcttacaaag

SEQ ID NO: 33 - DNA, 32 bp, Artificial Sequence
  Primer
tctagttctctggtcagtatcagcaataacag

SEQ ID NO: 34 - DNA, 28 bp, Artificial Sequence
  Primer
gaccagagaactagaaagactggtgagg

SEQ ID NO: 35 - DNA, 29 bp, Artificial Sequence
  Primer
caacaagaaaacctcagtcaaagtggagg

SEQ ID NO: 36 - DNA, 25 bp, Artificial Sequence
  Primer
gaggttttcttgttgcaagaggctc

SEQ ID NO: 37 - DNA, 28 bp, Artificial Sequence
  Primer
agccaagtatggagttctctgcaacaag

SEQ ID NO: 38 - DNA, 29 bp, Artificial Sequence
  Primer
tccatacttggctagatccagagctactc

SEQ ID NO: 39 - DNA, 25 bp, Artificial Sequence
  Primer
gtttgtgtcgtgaccagcaatgaac

SEQ ID NO: 40 - DNA, 27 bp, Artificial Sequence
  Primer
tcacgacacaaacttggctaacttgtc

SEQ ID NO: 41 - DNA, 25 bp, Artificial Sequence
  Primer
cttgagcggccgcttattcagtaac

SEQ ID NO: 42 - DNA, 26 bp, Artificial Sequence
  Primer
tctagttctctggtcaacatcagccc

SEQ ID NO: 43 - DNA, 28 bp, Artificial Sequence
  Primer
gaccagagaactagattgactggtgacg

SEQ ID NO: 44 - DNA, 25 bp, Artificial Sequence
  Primer
caacaagaaaacctcggaaacagtg

SEQ ID NO: 45 - DNA, 29 bp, Artificial Sequence
  Primer
gaggttttcttgttggagtacgctcaagg

SEQ ID NO: 46 - DNA, 28 bp, Artificial Sequence
  Primer
agccaagtatggagtcttagccatcaag

SEQ ID NO: 47 - DNA, 23 bp, Artificial Sequence
  Primer
tccatacttggcttccagaaagg

SEQ ID NO: 48 - DNA, 24 bp, Artificial Sequence
  Primer
gtttgtgtcgtgagcagccaaaac

SEQ ID NO: 49 - DNA, 26 bp, Artificial Sequence
  Primer
tcacgacacaaacttggctaatttgg

SEQ ID NO: 50 - DNA, 21 bp, Artificial Sequence
  Primer
cttgagcggccgcttatggtc

SEQ ID NO: 51 - DNA, 24 bp, Artificial Sequence
  Primer
tctagttctctggtcgatgtcagc

SEQ ID NO: 52 - DNA, 28 bp, Artificial Sequence
  Primer
gaccagagaactagattgactggtcagg

SEQ ID NO: 53 - DNA, 26 bp, Artificial Sequence
  Primer
caacaagaaaacctcacccaaagtag

SEQ ID NO: 54 - DNA, 25 bp, Artificial Sequence
  Primer
gaggttttcttgttgcagaactccc

SEQ ID NO: 55 - DNA, 28 bp, Artificial Sequence
  Primer
agccaagtatggagtcttagccatcaag

SEQ ID NO: 56 - DNA, 27 bp, Artificial Sequence
  Primer
tccatacttggctagacacaagggtac

SEQ ID NO: 57 - DNA, 25 bp, Artificial Sequence
  Primer
gtttgtgtcgtgaccacccaagaac

SEQ ID NO: 58 - DNA, 25 bp, Artificial Sequence
  Primer
tcacgacacaaacattgctaacatc

SEQ ID NO: 59 - DNA, 20 bp, Artificial Sequence
  Primer
cttgagcggccgcttagatg

SEQ ID NO: 60 - DNA, 26 bp, Artificial Sequence
  Primer
tctagttctctggtcaacatcagccc

SEQ ID NO: 61 - DNA, 28 bp, Artificial Sequence
  Primer
gaccagagaactagaaagactggtgagg

SEQ ID NO: 62 - DNA, 26 bp, Artificial Sequence
  Primer
caacaagaaaacctcagccaaagtag

SEQ ID NO: 63 - DNA, 23 bp, Artificial Sequence
  Primer
gaggttttcttgttggagcacgc

SEQ ID NO: 64 - DNA, 28 bp, Artificial Sequence
  Primer
agccaagtatggagttctggacatcaag

SEQ ID NO: 65 - DNA, 24 bp, Artificial Sequence
  Primer
tccatacttggctaagcacaacgg

SEQ ID NO: 66 - DNA, 22 bp, Artificial Sequence
  Primer
gtttgtgtcgtgaccagcaatg

SEQ ID NO: 67 - DNA, 25 bp, Artificial Sequence
  Primer
tcacgacacaaacattgctaacatc

SEQ ID NO: 68 - DNA, 20 bp, Artificial Sequence
  Primer
cttgagcggccgcttatggc

(19) United States
(12) Patent Application Publication
(10) Pub. No.: US 2024/0158769 A1
(43) Pub. Date: May 16, 2024

(54) Thermostable Phytase Chimera

(71) Applicant: RHEINISCH-WESTFÄLISCHE TECHNISCHE HOCHSCHULE (RWTH) AACHEN, Aachen (DE)

(72) Inventors: Anna Joelle Ruff, Charneux Herve (BE); Ulrich Schwaneberg, Kelmis-Hergenrath (BE); Mehdi Davaridolatabadi, Aachen (DE); Stefanie Hamer-Timmermann, Wedel (DE); Dennis Jungherz, Köln (DE)

(21) Appl. No.: 18/254,532

(22) PCT Filed: Nov. 30, 2021
(86) PCT No.: PCT/EP2021/083564

§ 371 Date: Jan. 26, 2024

(30) Foreign Application Priority Data
Dec. 1, 2020 (DE) 10 2020 131 856.1

(51) Int. Cl. C12N 9/16; C40B 30/08

(52) U.S. Cl. CPC C12N 9/16; C12Y 301/03008; C40B 30/08

(57) ABSTRACT

A phytase chimera, a nucleic acid having a nucleic acid sequence encoding such a phytase chimera, a vector having such a nucleic acid, a host cell having such a nucleic acid or such a vector, and the use of such a phytase chimera in food and animal feed production.

### TECHNICAL FIELD

The invention lies in the field of industrially usable enzymes, in particular phytases. More specifically, the present invention relates to a phytase chimera. Furthermore, the invention relates to a nucleic acid with a nucleic acid sequence encoding such a phytase chimaera, a vector with such a nucleic acid, a host cell with such a vector and the use of such a phytase chimaera in animal feed production, in particular as an animal feed additive or for obtaining phosphorus from de-oiled seeds.

### BACKGROUND TO THE INVENTION

Phytases hydrolyze sequentially phosphates from phytate (inositol-1,2,3,4,5,6-hexakisphosphate, IP6, InsP6), which is the main phosphorus storage form in plant seeds and is quite stable against abiotic degradation. Seeds, de-oiled seeds, grains and plants are basic components of animal feed. Seeds and grains often contain more than 1% phytate. Phosphorus utilization from phytate-rich feed is very inefficient for monogastric animals with low endogenous hydrolysis rates in the digestive system, such as fish, poultry or pigs. Phytate is also known as an antinutritional factor due to its strong chelating effect, because IP6 and its lower phosphorylated intermediates reduce the bioavailability of minerals and proteins.

Phytases are mainly used in food and animal feed production and are therefore of particular industrial importance. Among other things, phytases are added to animal feed as an additive, where they make the phosphorus present available to the animal organism. Phytases are added to the feed of animals with low endogenous phytase activity in order to improve animal productivity, reduce the amount of mineral phosphorus in feed and reduce the phosphate load on the environment, as the phytate-rich excretions are usually spread on fields.

According to statistics, the market for phytases in the animal feed production sector amounted to around USD 437 million in 2018 and is forecast to grow by around 6% annually until 2025. With the increasing acceptance of pets and especially farm animals, rising disposable income and steadily growing commercial livestock farming, the demand for animal feed will continue to increase in the coming years. In addition, the demand for meat has increased in recent years due to the growing population and producers and consumers have become more aware of environmental protection and sustainability. However, demand for phytases is expected to increase, particularly as the new Fertilizer Guideline stipulates a reduction in nitrogen and phosphorus/phosphate. In order to comply with the values specified in the Fertilizer Guideline, farmers are dependent on feeding in line with requirements. The production of pelleted feed poses particular challenges in connection with the desire to add phytases to the feed. Very high temperatures (>70° C.) are reached for a short time in the pelleting step. Only a few enzymes can withstand such high temperatures. There is therefore a need for thermostable phytases.

The recombination of genes with low sequence identities (distantly related family members) has the potential to identify chimeric enzymes that combine different properties in a single enzyme. For phytases, thermal stability would be a desirable property to withstand the high temperatures during feed pelleting. However, specific activity is a property that should not be neglected, as the profitability of phytases in animal feed applications also depends on it. Therefore, one challenge in improving the thermal stability of a protein is to maintain its specific activity at the same time. Rigid protein structures may in principle lead to improved thermal stability, but at the same time are associated with less flexibility in the active site and reduced specific activity. Therefore, a particular challenge is to achieve improved thermal stability without sacrificing specific activity.

The primary task of the present invention was therefore to provide a thermostable phytase. If possible, the phytase should not only have good thermostability, but also good specific activity.

This task is solved by a phytase chimera as described herein. A phytase chimera according to the present invention comprises in particular an amino acid sequence according to SEQ ID NO:1 or SEQ ID NO:2, or an amino acid sequence having at least 80% sequence identity thereto.

Using computer-aided analysis with multiple sequence alignment and homology modeling, the inventors succeeded in identifying various fragments of different phytase genes (sequence identities 31 to 64%) and recombining them by phosphorothioate-based cloning using the PTRec method. By combinatorial recombination, phytase chimeras were obtained, which surprisingly turned out to be functional. The functional variants of the phytase chimera have an amino acid sequence according to SEQ ID NO:1 or SEQ ID NO:2, or an amino acid sequence with at least 80% sequence identity to these. It is particularly noteworthy that all tested variants covered by the above definition had a T50₁₅ value of 50° C. to 65° C. This means that the variants had a residual activity of 50% after exposure for 15 min at 50° C. to 65° C. It should be noted that a simplified assay was used for this, which was based on measurements in the supernatant of a screening library and it can be assumed that the thermostability of the pure enzyme fraction is significantly higher. This was confirmed by measurements of selected variants of the phytase chimera according to the invention. These showed a residual activity of at least 65% even after 30 min at 90° C., while these variants had a T50₁₅ value of 61.8 or 63.8° C. in the simplified assay (i.e. after exposure for 15 min at 61.8° C. or 63.8° C. they had 50% residual activity). Furthermore, all variants showed a high specific activity. As described in more detail elsewhere, the phytase chimera with an amino acid sequence according to SEQ ID NO:1 or SEQ ID NO:2 were particularly thermostable and active, which accordingly represents a particularly preferred embodiment of the invention.

The amino acid sequence according to SEQ ID NO:1 denotes a variant that is also referred to here as PTRec 77. The amino acid sequence according to SEQ ID NO:2 denotes a variant that is also referred to here as PTRec 74. Both variants are composed of sequence segments of phytases from Citrobacter braakii, Hafnia alvei and Yersinia mollaretii. As already described, these two variants are particularly thermostable and active variants, which makes them particularly suitable for use as feed additives.

The problem is further solved by a nucleic acid having a nucleic acid sequence encoding a phytase chimera as described herein, a vector having a nucleic acid as described herein, a host cell having a vector as described herein, and the use of a phytase chimera as described herein in food and animal feed production, in particular for use as a feed additive.

Further aspects, embodiments and advantages of the invention are apparent from the detailed description and the experimental part together with the figures and the claims. The sequence listing attached to the present application is hereby incorporated by reference.

### DEFINITIONS

The term “phytase” refers to a protein with phytase activity, i.e. with the ability to sequentially hydrolyze phytic acid and thus release the bound phosphate. More precisely, the products myo-inositol pentakisphosphate (IP5) (as well as the other lower inositols IP4, IP3, IP2, IP1 and IP) and phosphate are formed from the substrates myo-inositol hexakisphosphate (IP6) and water in an enzyme-catalyzed reaction. According to the enzyme classification, phytases (EC 3.1.3.8) are assigned to the phosphatases.

In the present case, the term chimera refers to a recombinant phytase derived from phytases (or parts thereof) of different origin. Since the phytase chimera according to the invention was created by recombination of phytases from different organisms, it is reasonable to assume that the chimera does not occur in identical form in nature. Although based on naturally occurring phytases, it is possible that the sequence of the phytase chimera has further modifications such as substitutions, additions or deletions, and/or additional sequence segments such as signal peptides, compared to the corresponding sequence segments occurring in nature from which the phytase chimera is derived.

Preferably, a chimera refers to a recombinant phytase composed of coherent sequence segments of two, three or more (preferably three) phytases of different origin or composed of coherent sequence segments each having at least 80%, preferably at least 81%, more preferably at least 82%, more preferably at least 83%, more preferably at least 84%, more preferably at least 85%, more preferably at least 86%, more preferably at least 87%, further preferably at least 88%, further preferably at least 89%, further preferably at least 90%, further preferably at least 91%, further preferably at least 92%, further preferably at least 93%, further preferably at least 94%, further preferably at least 95%, further preferably at least 96%, further preferably at least 97%, further preferably at least 98%, most preferably at least 99% sequence identity to coherent sequence segments of two, three or more phytases of different origin. It is further preferred that the coherent sequence segments each have a length of at least 30, preferably at least 35, more preferably at least 40 contiguous amino acids.

The chimaeras according to the invention are preferably composed of coherent sequence segments of phytases from Citrobacter braakii, Hafnia alvei and Yersinia mollaretii or of coherent sequence segments which each comprise at least 80%, preferably at least 81%, further preferably at least 82%, further preferably at least 83%, further preferably at least 84%, further preferably at least 85%, further preferably at least 86%, further preferably at least 87%, further preferably at least 88%, further preferably at least 89%, further preferably at least 90%, further preferably at least 91%, further preferably at least 92%, further preferably at least 93%, further preferably at least 94%, further preferably at least 95%, further preferably at least 96%, further preferably at least 97%, further preferably at least 98%, most preferably at least 99% sequence identity to coherent sequence segments of phytases from Citrobacter braakii, Hafnia alvei and Yersinia mollaretii.

The coherent sequence segment of the phytase from Citrobacter braakii preferably has a length of at least 30, preferably at least 35, more preferably at least 40 contiguous amino acids. The coherent sequence segment of the phytase from Hafnia alvei preferably has a length of at least 70, preferably at least 75, 80, 85, 90, 95, 100, 105, particularly preferably at least 110 contiguous amino acids. The coherent sequence segment of the phytase from Yersinia mollaretii preferably has a length of at least 70, preferably at least 75, 80, 85, 90, 95, 100, 105, particularly preferably at least 110 contiguous amino acids.

Amino acid sequence identity is understood here to mean the percentage of amino acids in a sequence of a candidate protein that occurs in an identical manner (i.e. exactly in this order) in the sequence of a specific protein (here, for example, phytase chimera with an amino acid sequence according to SEQ ID NO:1).

The same applies to a nucleic acid sequence identity: Nucleic acid sequence identity is understood here as the percentage of nucleotides in the sequence of a candidate nucleic acid that occurs in an identical manner (i.e. exactly in this order) in the sequence of a specific nucleic acid (here, e.g., a nucleic acid with a sequence according to SEQ ID NO:4).

If the amino acid sequence identity or nucleic acid sequence identity is related to a phytase chimera according to the invention or a nucleic acid encoding therefor, preferably only the sequence segment or only the sequence segments relating to the phytase are taken into account. In particular, sequence segments which relate, for example, to signal peptides or other peptides fused with the phytase, which simplify the production of the phytase, are not taken into account.

Amino acid sequence identities and nucleic acid sequence identities specified here are based on sequence alignments evaluated using the EMBOSS Water Pairwise Sequence Alignments (nucleotides) program (http://www.ebi.ac.uk/Tools/psa/emboss_water/nucleotide.html) for nucleic acids and EMBOSS Water Pairwise Sequence Alignments (protein) program (http://www.ebi.ac.uk/Tools/psa/emboss_water/) for amino acids using the default parameters defined by EMBL-EBI. These parameters are (i) for amino acid sequences: Matrix=BLOSUM62, gap open penalty=10 and gap extend penalty=0.5 and (ii) for nucleic acid sequences at: Matrix=DNAfull, gap open penalty=10 and gap extend penalty=0.5. The above programs are provided by the European Molecular Biology Laboratory (EMBL) European Bioinformatics Institute (EBI) for local sequence alignments and use a modified Smith-Waterman algorithm (see http://www.ebi.ac.uk/Tools/psa/ and Smith, T.F. & Waterman, M.S. “Identification of common molecular subsequences” Journal of Molecular Biology, 1981 147 (1):195-197).

The term “sequence segment” as used herein refers to a coherent/coherent sequence of multiple amino acids or nucleotides. In particular, a sequence segment refers to a partial sequence of a (whole) sequence. This (overall) sequence can represent the sequence of a protein (here, for example, a phytase chimaera) or a nucleic acid (here, for example, a nucleic acid encoding for a phytase chimaera), or also the sequence of a fusion protein (here, for example, a phytase chimaera with a signal peptide) or a corresponding nucleic acid (for example, a nucleic acid coding for a phytase chimaera and a signal peptide).

The term “amino acid position” in connection with a number n denotes the nth amino acid in an amino acid sequence, with counting starting from the 5′-end.

The “specific phytase activity”, as used here, refers to the mass-specific enzyme activity and has the dimension U/mg. One unit (U) is defined as the amount of enzyme required to release 1 μM of inorganic orthophosphate per minute from sodium phytate.

Where reference is made in this text to activity values and residual activities and nothing else is stated, these are the values obtained when the enzyme (enzyme concentration 50 μg/mL) is diluted (final enzyme concentrations of 55-110 ng/mL), cooled to ice after heat treatment if necessary, and the phytase activity is determined on the basis of a reaction of 0.4 mM IP6 in 50 mM NaOAc, pH 4.9, at 60° C. over a period of 6 min.

The terms “protein” or “peptide” are used interchangeably herein without reference to a specific length. In principle, unless further specified, all natural and non-natural proteins, peptides and polypeptides as well as modified proteins, peptides and polypeptides are included. In this context, the term “modified” refers to any chemical modification, irrespective of how the modification was induced.

### BRIEF DESCRIPTION OF THE FIGURES

FIG. 1 shows the thermostability of 16 phytase chimera variants identified in the screening of a PTRec library with the highest residual activity after 15 min at 60° C. The thermostability was investigated between 50 and 70° C. by exposing the variants to the respective temperature for 15 min and then determining the residual activity. Approximate T50₁₅ values were calculated on the basis of these data. The Chimera DJ 11 and DJ 14 are also referred to here as PTRec 77 and PTRec 74.

FIG. 2 shows the determination of residual activities of selected phytase chimera in comparison to the parental phytases after incubation at 90° C. for 30 and 60 minutes (thermostability). After incubation, the treated enzymes were placed on ice for 30 minutes and the residual activity was then determined. It was found that the two phytase chimaeras examined in more detail (PTRec 74 and PTRec 77) had a higher residual activity compared to all parental enzymes.

### DETAILED DESCRIPTION

The present disclosure generally relates to a phytase chimera having an amino acid sequence comprising sequence segments of the three phytases from Citrobacter braakii (Cb), Hafnia alvei (Ha) and Yersinia mollaretii (Ym), or having an amino acid sequence with at least 80%, preferably at least 85%, more preferably at least 90%, more preferably at least 95%, most preferably at least 97% sequence identity thereto. The phytases from Cb, Ha and Ym have an amino acid sequence according to SEQ ID NO:5, SEQ ID NO:6 or SEQ ID NO:7.

In a first aspect, the invention relates to a phytase chimera having an amino acid sequence according to SEQ ID NO:1 or SEQ ID NO:2, or an amino acid sequence having at least 80% sequence identity thereto. Preferably, the sequence identity of the phytase chimera according to the invention is at least 82%, more preferably at least 84%, more preferably at least 86%, more preferably at least 88%, more preferably at least 90%, more preferably at least 91%, more preferably at least 92%, more preferably at least 93%, more preferably at least 94%, more preferably at least 95%, more preferably at least 96%, more preferably at least 97%, more preferably at least 98%, most preferably at least 99%.

In a further embodiment of the invention, the phytase chimera has an amino acid sequence according to SEQ ID NO:1, SEQ ID NO:2, SEQ ID NO:8 (here also referred to as variant DJ 15), SEQ ID NO:9 (here also referred to as variant DJ 2), SEQ ID NO:10 (here also referred to as variant DJ 19) or SEQ ID NO:11 (also referred to herein as variant DJ 24), or an amino acid sequence having at least 90%, preferably at least 91%, more preferably at least 92%, more preferably at least 93%, more preferably at least 94%, more preferably at least 95%, more preferably at least 96%, more preferably at least 97%, more preferably at least 98%, most preferably at least 99% sequence identity thereto.

In one embodiment of the invention, the amino acid sequence comprises a sequence segment with amino acids 92 to 415 of the sequence according to SEQ ID NO:1, or a sequence segment with at least 90% sequence identity thereto. Preferably, the sequence identity to the aforementioned sequence segment is at least 91%, further preferably at least 92%, further preferably at least 93%, further preferably at least 94%, further preferably at least 95%, further preferably at least 96%, further preferably at least 97%, further preferably at least 98%, most preferably at least 99%. This sequence segment was present in all particularly thermostable and at the same time active variants of the phytase chimera according to the invention.

Particularly preferred are phytase chimeras with an amino acid sequence according to SEQ ID NO:1 or SEQ ID NO:2. As already mentioned at the beginning, the amino acid sequence according to SEQ ID NO:1 denotes a variant, which is also referred to herein as PTRec 77, and the amino acid sequence according to SEQ ID NO:2 denotes a variant, which is also referred to herein as PTRec 74. Both variants are composed of sequence segments of phytases from Cb, Hf and Ym as described herein. More precisely, PTRec 77 is composed of 60% Ha phytase, 29.8% Ym phytase and 10.2% Cb phytase. PTRec 74 is composed of 35.2% Ha phytase, 54.6% Ym phytase and 10.2% Cb phytase. Due to their particularly good thermostability and specific activity, these variants are particularly suitable for the production of animal feed.

As already mentioned at the beginning, the phytase chimera according to the invention is particularly thermostable. In particular, the phytase chimera according to the invention has a phytase stability which is characterized by a T50₁₅ value, preferably a T50₃₀ value or a T50₆₀ value of at least 50° C., preferably at least 60° C., more preferably at least 70° C., more preferably at least 80° C., most preferably at least 90° C.

Here, the T50₁₅ value, the T50₃₀ value and the T50₆₀ value are defined as the temperatures at which a 50 μg/mL preparation of the phytase chimera still has 50% residual activity after 15, 30 or 60 minutes of exposure compared to a correspondingly long exposure at 25° C.

A further advantage already mentioned, which is associated with the phytase chimera according to the invention, relates to the relatively high specific phytase activity. In particular, the phytase chimera according to the invention has a specific phytase activity of at least 500 U/mg, preferably at least 600 U/mg, more preferably at least 700 U/mg, more preferably at least 750 U/mg, more preferably at least 800 U/mg, more preferably at least 850 U/mg, more preferably at least 900 U/mg, more preferably at least 950 U/mg, most preferably at least 1000 U/mg. As will be known to those skilled in the art, activity determinations may vary not only depending on the amino acid sequence but also depending on the host used to produce the phytase chimera and the measurement conditions used to determine the activity.

The phytase chimera according to the invention may comprise functional peptides. In the present context, a functional peptide refers to a peptide that has functions other than phytase activity. In particular, it is preferred that the phytase chimera according to the invention comprises a signal peptide so that the phytase chimera according to the invention can be secreted after microbial production and removed from the cell supernatant. For this purpose, the sequences specific for the respective production organism are known to the person skilled in the art.

In one embodiment of the invention, it is a phytase chimera produced or producible using Pichia pastoris (also known as Komagataella phaffii). In particular, it is a phytase chimera having a glycosylation pattern resulting from production by Pichia pastoris. The thermostability of the phytase according to the invention can be additionally influenced by removing or adding glycosylation sites.

In another aspect, the present invention relates to a nucleic acid having a nucleic acid sequence encoding a phytase chimera as described herein. Specific nucleic acids are those having a sequence according to SEQ ID NO: 3 encoding the phytase chimera referred to herein as PTRec 77, and SEQ ID NO: 4 encoding the phytase chimera referred to herein as PTRec 74, or a nucleic acid sequence having at least 90% sequence identity to the sequence according to SEQ ID NO: 3 or SEQ ID NO: 4. In accordance with the present invention, a defined nucleic acid includes not only the identical nucleic acid, but also any other minor base variations, including in particular substitutions in bases leading to a synonymous codon (a different codon specifying the same amino acid residue) as a result of the degenerate code. A nucleic acid sequence as disclosed herein also includes the complementary sequence to any specified single-stranded sequence.

The nucleic acid according to the invention may advantageously be included in a suitable expression vector for expressing the enzyme encoded thereby in a suitable host. Accordingly, another aspect of the present invention relates to a vector comprising a nucleic acid as described herein.

An expression vector according to the invention includes a vector comprising a nucleic acid according to the invention which is functionally linked to regulatory sequences, such as promoter regions, capable of effecting expression of the nucleic acid according to the invention. The term “functionally linked” refers to a juxtaposition in which the described components are in a relationship that allows them to fulfill their function in the desired manner.

The vectors can be, for example, plasmid, virus or phage vectors, which are provided with an origin of replication, possibly a promoter for the expression of the nucleotide and possibly a regulator of the promoter. The vectors may contain one or more selectable markers, such as zeocin resistance.

Regulatory elements required for expression include promoter sequences to bind RNA polymerase and transcription initiation sequences for ribosome binding. For example, a bacterial expression vector may include a promoter such as a lac promoter and, for translation initiation, a Shine-Dalgarno sequence and a AUG start codon. Similarly, an eukaryotic expression vector may include a heterologous or homologous promoter for RNA polymerase II, a downstream polyadenylation signal, start codon AUG and a termination codon for ribosome detachment. Such vectors can be obtained commercially or assembled from described sequences by methods generally known in the field.

Such vectors can be transformed into a suitable host cell to provide a vector or genome integrated part of the vector containing the nucleic acid as described herein for the expression of an enzyme according to the invention. Thus, according to a further aspect, the invention provides a host cell comprising a nucleic acid as described herein or comprising a vector as described herein.

The present invention also relates to a method for producing a phytase chimera according to the invention, which comprises culturing a host cell transformed, transfected or infected with a vector as described herein, under conditions to effect expression by the vector of a nucleic acid sequence encoding the phytase chimera, and recovering the expressed phytase chimera.

Finally, the present invention relates to the use of a phytase chimera as described herein in food and animal feed production. In particular, the phytase chimera according to the invention is suitable as an additive in food and animal feed production, in particular comprising a step which takes place at an elevated temperature such as for example at least 37° C., preferably at least 42° C., more preferably at least 50° C., more preferably at least 60° C., more preferably at least 70° C., more preferably at least 80° C., most preferably at least 90° C. An example of such a step is feed pelletization. Another example relates to the recovery of phosphorus from, for example, de-oiled seeds. Here, the recovery of phosphorus from deoiled seeds preferably takes place at a temperature of at least 37° C., preferably at least 42° C. The aforementioned uses are described in more detail in the German patent application 10 2020 200 670.9 filed with the German Patent and Trade Mark Office on Jan. 21, 2020 with the title “ Verfahren zur Bereitstellung von Phosphat aus einer Phytat-haltigen Biomasse, Phytat- und Phosphat-reduzierte Biomasse und Verwendungen hiervon” (Process for providing phosphate from a phytate-containing biomass, phytate- and phosphate-reduced biomass and uses thereof), which is hereby incorporated by reference in its entirety for this purpose.

Where reference is made to specific SEQ ID NOs in this text, the following sequences are meant:
SEQ ID NO:1—Phytase chimera PTRec 77
SEQ ID NO:2—Phytase chimera PTRec 74
SEQ ID NO:3—Gene of the phytase chimera PTRec 77
SEQ ID NO:4—Gene of the phytase chimera PTRec 74
SEQ ID NO:5—Citrobacter braakii phytase (Cb phy, AAS45884.1) with AA substitutions compared to WT: I219V, E261Y, V262L
SEQ ID NO:6—Hafnia alvei phytase (Ha phy, 4ARS_A) with AA substitutions compared to WT: I223V, I266L, AA substitutions compared to WT (inserted during gene synthesis): S186G
SEQ ID NO:7—Yersinia mollaretii phytase variant M1 (Ym phy, AEI69378.1) with AA substitutions compared to WT: I225V, I268L, AA substitutions of variant M1 compared to WT: D35N, TOOK, K122E, Q1705, V281M
SEQ ID NO:8—Phytase chimera variant “DJ 15”
SEQ ID NO:9—Phytase chimera variant “DJ 2”
SEQ ID NO:10—Phytase chimera variant “DJ 19”
SEQ ID NO:11—Phytase chimera variant “DJ 24”
SEQ ID NOs: 12 to 68: Primers used for the phytase library

The aforementioned sequences and the associated sequence listing are considered part of the description.

The present invention is now further described by the following example and the accompanying drawings.

### Example

In the following example, variants of the phytase chimera according to the invention are characterized which were obtained by recombination of parts of the phytase genes from C. braakii (Cb phy), H. alvei (Ha phy) and Y. mollaretii (Ym phy) using an episomal P. pastoris expression system and identified by means of functional screening for improved thermal stability. Details of the recombination, screening and presentation of the variants characterized here are not described below, but are within the competence of the skilled person. In addition, reference is made to a post-published paper by the inventors (Herrmann et al. Generation of phytase chimeras with improved thermal stability by phosphorothioate-based DNA recombination. In preparation).

Improved variants were characterized in detail with respect to specific activity on phytate (IP6), temperature and pH optima, melting temperature and thermal stability at 90° C. to demonstrate the hidden potential of tailor-made phytase chimera.

### 1 Experimental Part

### 1.1 Material

All chemicals used in this study were of analytical or higher quality and were purchased from AppliChem (Darmstadt, Germany), Sigma-Aldrich Chemie (Taufkirchen, Germany) or Carl Roth (Karlsruhe, Germany). The expression strain Pichia pastoris (Komagataella phathi) BSYBG11 and the plasmids pBSYAG1Zeo and BSY3S1Z were purchased from bisy e.U. (Hofstätten/Raab, Austria).

The phytase library was created on the basis of the following phytase genes using the primers specified in the sequence listing (SEQ ID NOs: 12 to 68) and the crossover sequences also specified below:

Citrobacter braakii phytase (Cb phy, AAS45884.1), whereby the following amino acid substitutions were present compared to the wild type: I219V, E261Y, V262L (SEQ ID NO:5);

Hafnia alvei phytase (Ha phy, 4ARS_A), whereby the following amino acid substitutions were present compared to the wild type: I223V, I266L-S186G (inserted during gene synthesis) (SEQ ID NO:6);

Yersinia mollaretii phytase variant M1 (Ym phy, AEI69378.1), whereby the following amino acid substitutions were present compared to the wild type: I225V, I268L. Compared to the wild type, the M1 variant has the following amino acid substitutions: D35N, T6OK, K122E, Q170S, V281M (SEQ ID NO:7):

Crossover sequences: QRTR, EVFL, PYLA, HDTN.

### 1.2 Methods

### 1.2.1 Model Reaction for Phytase Characterization

The conversion of IP6 for enzyme characterization was performed in PCR tubes in a PCR cycler (Bio-Gener GET3XG, Hangzhou, China). The enzyme dilution (60 μL) and IP6 (80 μL, 0.4 mM) were pre-warmed in separate PCR tubes for 2 minutes at the desired assay temperature. All components were dissolved in 50 mM buffer at the optimal pH. The reaction was started by adding IP6 (60 μL, 0.4 mM) to the enzyme tube. After 6 min, the reaction was stopped by adding TCA (15%, 60 μL), mixed thoroughly and then subjected to phosphate quantification.

### 1.2.2 Spectrophotometric Quantification of Phosphate

Phosphate was quantified using the molybdenum blue reaction. 150 μL of the TCA-stopped phytase reaction mixture was mixed with 50 μL of ammonium molybdate color developing solution in an MTP according to Herrmann et al. (Herrmann, K.R., Ruff, A.J., Infanzon, B., Schwaneberg, U., 2019. Phytase-Based Phosphorus Recovery Process for 20 Distinct Press Cakes Kevin R. Herrmann, Anna Joëlle Ruff, and Ulrich Schwaneberg, ACS Sustainable Chem. Eng. 2020, 8, 9, 3913-3921, Publication Date: February 11, 2020, https://doi.org/10.1021/acssuschemeng.9b07433).

### 1.2.3 Determination of the pH Optimum

The determination of the optimum pH value was carried out in the microtiter plate (MTP). The enzyme dilution (50 μL) was mixed with IP6 (50 μL, 0.4 mM) and further treated as described in section 1.2.1. The activity was tested in a pH range from 2.2 to 5.6. In the pH range from 2.2 to 3.3 a glycine-HCl buffer (50 mM) was used, above pH 3.3 a NaOAc buffer (50 mM) was used.

### 1.2.4 Determination of Thermostability

To determine the thermal stability of the purified enzymes, a phytase dilution (80 μL of 50 μg/mL) was incubated in PCR tubes (30 to 60 min; 90 to 95° C.) using a PCR cycler (BioGener GET3XG, Hangzhou, China). After incubation (30 min on ice), further dilutions were prepared and enzymatic conversion was performed according to the model reaction.

### 1.2.5 Melting Temperature

The determination of the melting temperature (Tm) was performed using the Applied Biosystems® Protein Thermal Shift™ dye kit according to the manufacturers instructions (Thermo Fisher Scientific, Darmstadt, Germany). 5 μg protein per reaction dissolved in NaOAc (50 mM, pH 5) was analyzed in an Applied Biosystems® Step One Plus real-time PCR system (Carlsbad, USA).

### 1.2.6 Determination of Protein Glycosylation

The protein glycosylation assay was performed with Promega's endoglycosidase H (Endo H) (Promega GmbH, Mannheim, Germany) according to the manufacturers protocol. 10 μg protein was mixed with Endo H (2 μL, 1000 units) and the reaction was incubated (18 h, 37° C.).

### 2. Results and Discussion

### 2.1 Library Screening

The library screening for thermal stability yielded 16 promising variants, which were further analyzed. A more detailed analysis of the 16 variants was performed by determining the residual activity on the natural substrate IP6 after temperature treatment and subsequent evaluation of the T50 based on the AMol assay, which provides an approximate but sufficiently accurate value. The AMol assay is based on the molybdenum blue reaction, in which ammonium molybdate reacts with free phosphate (PO₄³⁻) to form molybdenum blue. This blue coloration can be measured spectroscopically. The more phosphate in solution, the bluer the color.

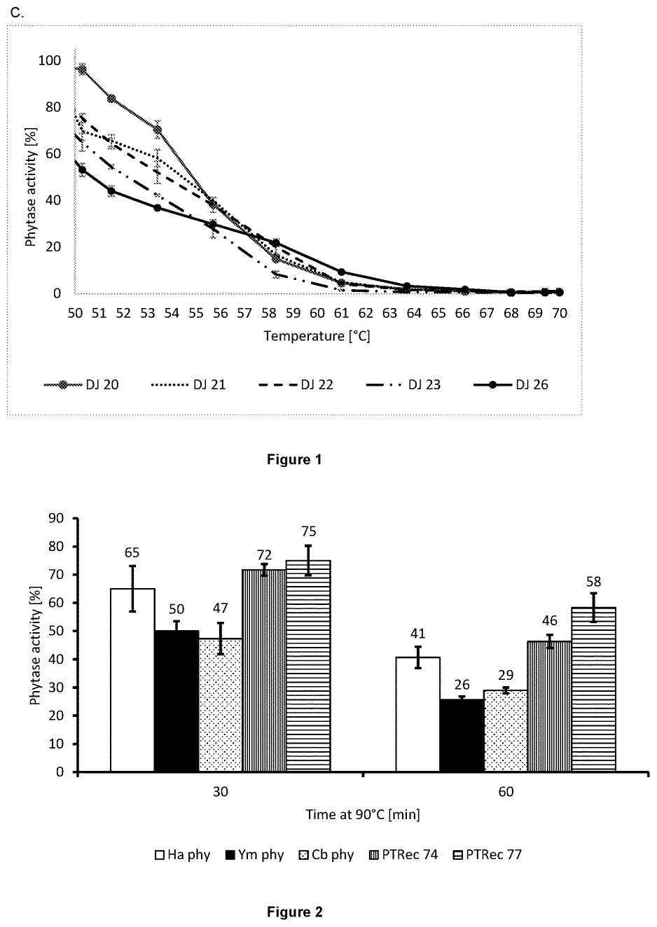

The T50₁₅ is defined in this context as the temperature at which the phytase loses 50% of its activity after 15 minutes of heat treatment compared to incubation at RT (25° C.). The determination was carried out with non-purified enzymes. The lowest T50₁₅ was determined for DJ 26 at about 50° C., but four variants showed a T50₁₅ of even more than 60° C. and up to about 64° C. (see FIG. 1).

Ten variants (eight with the highest T50₁₅ and two lower ones) were then amplified by PCR and sequenced. The sequencing revealed that nine out of ten variants were chimaeras, which are composed of several fragments of different phytases (cf. Table 1).

TABLE 1
Fragment order of fragments A to E and origin of
ten phytase variants with high T50₁₅ values in the
P. pastoris culture supernatant found during screening
of PTRec DNA recombination libraries. The fragment
length is given as the number of amino acids (AA)
of the corresponding fragment. In addition, the T50₁₅ value
determined in the supernatant is indicated. Ha: Hafnia alvei phytase;
Ym: Yersinia mollaretii phytase, Cb: Citrobacter braakii phytase.

| | ~T₅₀ | A | B | C | D | E |
|---|---|---|---|---|---|---|
| Variant | [° C.] | 94 AA | 130 AA | 42 AA | 42 AA | 106 AA |
| PTRec 77 | 64 | Ha | Ym | Cb | Ha | Ha |
| | | 94 AA | 130 AA | 42 AA | 42 AA | 106 AA |
| DJ 12 | 64 | Ha | Ym | Cb | Ha | Ha |
| | | 95 AA | 130 AA | 42 AA | 42 AA | 106 AA |
| PTRec 74 | 62 | Ym | Ym | Cb | Ha | Ha |
| | | 94 AA | 129 AA | 42 AA | 42 AA | 106 AA |
| DJ 15 | 58 | Ha | Ha | Cb | Ha | Ha |
| | | 94 AA | 129 AA | 42 AA | 42 AA | 107 AA |
| DJ 16 | 58 | Ha | Ha | Ha | Ha | Ym |
| | | 95 AA | 130 AA | 42 AA | 43 AA | 107 AA |
| DJ 2 | 57 | Ym | Ym | Cb | Ym | Ym |
| | | 94 AA | 130 AA | 42 AA | 42 AA | 107 AA |
| DJ 19 | 56 | Ha | Ym | Cb | Ha | Ym |
| | | 95 AA | 130 AA | 42 AA | 42 AA | 107 AA |
| DJ 24 | 52 | Ym | Ym | Cb | Ha | Ym |
| | | 95 AA | 130 AA | 42 AA | 42 AA | 107 AA |
| DJ 25 | 52 | Ym | Ym | Cb | Ha | Ym |

Sequence identities were then determined using EMBOSS Water (https://www.ebi.ac.uk/Tools/psa/emboss_water/), on the one hand of the variants in relation to PTRec 74 and PTRec 77 and on the other hand of PTRec 74 and PTRec 77 in relation to the parental enzymes. The results are shown in Tables 2 and 3.

TABLE 2
Sequence identities (Seq. ident.) of some variants with a
T50₁₅ > 50° C. compared to PTRec 74 and 77.

| | Seq. Ident. for | Seq. Ident. for |
|---|---|---|
| | PTRec 74 [%] | PTRec 77 [%] |
| DJ 12* | 91.3 | 100.0 |
| DJ 15 | 74.9 | 83.7 |
| DJ 16 | 59.9 | 68.7 |
| DJ 2 | 82.5 | 73.7 |
| DJ 19 | 79.2 | 88.0 |
| DJ 24 | 88.0 | 79.2 |
| DJ 25 | 88.0 | 79.2 |

*Variant DJ 12 is identical to PTRec 77.
** Variants DJ 24 & DJ 25 are identical to each other.

TABLE 3
Sequence identities (Seq. ident.) of PTRec 74 and 77
compared to the 3 parental enzymes integrated in the
phytase chimera. In addition, the sequence identity
to the wild type (WT) of Ym phy was determined.

| | Seq. Ident. for | Seq. Ident. for |
|---|---|---|
| | PTRec 74 [%] | PTRec 77 [%] |
| Cb phy | 53.4 | 54.2 |
| Ha phy | 71.7 | 80.5 |
| Ym phy M1 | 79.1 | 70.4 |
| Ym phy WT (AEI 69378.1) | 77.7 | 69.7 |

The variants PTRec 77 and DJ 12 as well as the sequences of DJ 24 and DJ 25 showed an identical fragment arrangement. Fragment B of Ym phy, fragment C of Cb phy and fragment D of Ha phy were clearly overrepresented, while no fragment of Ec phy was observed in the chimera with the highest T50 value. The occurrence of many fragments of thermostable Ha phy in the variants with the highest thermal stability is striking. The most promising variants (PTRec 77 and DJ 12), which show the highest T50 values in the supernatant of the P. pastoris culture, have the same fragment composition (A: H. alvei, B: Y. mollaretii, C: C. braakii, D: H. alvei and E: H. alvei).

### 2.2 Characterization of Selected Phytases

Subsequently, the two most promising chimaeras (PTRec 77 and PTRec 74) were purified and characterized in comparison to their parental enzymes. PTRec 74 and PTRec 77 are composed of fragments of Cb phy, Ha phy and Ym phy. The only difference between the two chimeras is fragment A (Table 1).

After production in P. pastoris cells and protein purification, both a distinct band and smearing at higher molecular weights were observed. Treatment with endoglycosidase H (Endo H) removes the N-linked glycans and lowers the molecular weight, confirming glycosylation on all five parental enzymes. Overall, purities greater than 88% were achieved for all phytases after purification (data not shown).

The parental enzymes and the two chimaeras were then analyzed for pH and temperature optima, specific activity with respect to IP6, melting temperature and thermal stability. Data for some of these parental enzyme properties are already available in the literature, however, other expression hosts (such as B. subtilis or E. coli) were used, which may influence the enzymatic behavior, e.g. due to lack of glycosylation. Therefore, all the above properties were analyzed for the two chimaeras and all three parental enzymes secreted by P. pastoris.

The determination of pH value and temperature optima revealed a pH optima for Ym and Ha phy at pH 4.2 and the chimera show an optimum at pH 4.9. The optimum pH value of Cb phy is shifted into the acidic range at pH 3.3. The optimal temperature of Cb phy also differs significantly from all other phytases at 35° C. Ym phy has an optimum temperature of 55° C. and Ha phy, PTRec 74 and PTRec 77 have an optimum at 60° C. Both chimera show a very similar pH and temperature optimum, which is to be expected since their composition is identical except for fragment A. Fragment A obviously has no influence on these two properties. The data obtained for the parental enzymes Ha phy and Ym phy are in agreement with the literature data and only the optimal temperature of 35° C. for Cb phy differs significantly. An optimum temperature of 50° C. was reported for Cb phy. The fact that Citrobacter braakii YH-15 was used as expression host could explain the differences. A summary of all important enzyme properties that were determined is shown in Table 4.

TABLE 4
Summary of the investigated enzyme properties of the
parental enzymes and the recombinant phytase chimera

| | | | | Residual activity | specific |
|---|---|---|---|---|---|
| | | Tₒₚₜ | Tₘ | (60 min, 90° C.) | activity |
| | pHₒₚₜ | [° C.] | [° C.] | [%] | [U/mg] [symbol] |
| Cb phy | 3.3 | 35 | 56.9 ± 0.1 | 29 ± 1 | 959 ± 186 |
| Ha phy | 4.2 | 60 | 67.4 ± 0.1 | 41 ± 4 | 1529 ± 243 |
| Ym phy | 4.2 | 55 | 59.3 ± 0.1 | 26 ± 1 | 1515 ± 131 |
| PTRec 74 | 4.9 | 60 | 65.6 ± 0.2 | 46 ± 2 | 1209 ± 176 |
| PTRec 77 | 4.9 | 60 | 64.6 ± 0.1 & | 58 ± 5 | 1147 ± 179 |
| | | | 76.1 ± 0.1 | | |

[symbol]

one unit (U) is defined as the amount of enzyme required to release 1 μM of inorganic orthophosphate per minute from sodium phytate; the specific activity was determined for each enzyme at optimum temperature.

To investigate protein unfolding and determine the Tm of the enzymes, a thermoshift assay was performed (data not shown). For curve fitting of PTRec 77, the three-state model was used, while for all other samples the two-state model was applied. Cb phy not only has the lowest optimum temperature, but also the lowest melting temperature (57° C.). The second lowest melting temperature was observed for Ym phy (59° C.; in comparison: 64.5° C. for expression in E. coli), followed by Ha phy (67° C.) and the chimera PTRec 74 (66° C.). In contrast to all other enzymes tested, PTRec 77 shows two unfolding events with melting temperatures at 65° C. (Tm 1) and 76° C. (Tm 2). The two melting phases observed for PTRec 77 may be due to oligomerization, separate unfolding of different protein domains, or a different glycosylation pattern, resulting in two populations. Size exclusion chromatography (SEC) experiments were performed to test whether PTRec 77 forms a dimer. Two distinct peaks were observed (data not shown). Based on protein standards, the first, broader peak corresponds to proteins with a size of 80 kDa, while the second, sharper peak corresponds to proteins with a size of 50 kDa. Since a non-glycosylated homodimer of PTRec 77 would exceed 90 kDa, the SEC data do not indicate dimerization of PTRec 77 in the assay buffer. The untreated SEC fractions were analyzed by SDS-Page in comparison to deglycosylated (Endo H-treated) fractions (data not shown). The untreated samples of the first and second peak of the SEC experiment were approximately 90 and 55 kDa, respectively. Endo H treatment reduced the size of the proteins of the first peak to about 55 kDa, while the size of the proteins of the second peak was not affected. Therefore, the first peak is most likely a result of heterogeneous glycosylation of PTRec 77. Heterogeneity in the N-glycan pattern of secreted glycoproteins due to hypermannosylation is known for yeast, including P. pastoris. Following SEC, PTRec 77 SEC fractions 20 and 23 were analyzed by thermoshift assay to determine their melting temperatures (data not shown). Two separate unfolding events were further observed for both fractions. Of note, hypermannosylation in SEC fraction 20 leads to an increase in Tm2 (>3° C.) but has no effect on Tm1 compared to SEC fraction 23 (data not shown).

In summary, it can be said that the two melting phases observed for PTRec 77 are most likely due to the unfolding of different protein domains and not to the dissociation of a dimer. The lesson learned from this is that enzymes (chimera) can be endowed with new properties (e.g. two separate melting points) by recombination of different structural elements compared to the parental enzymes.

Improvements in thermal stability are often associated with reduced specific activity due to stiffening of the protein. The analysis of the specific activity with respect to IP6 revealed activities of about 960 to 1530 U/mg for the parental enzymes and more than 1100 U/mg for the chimera (Table 1). The more thermostable variant PTRec 77 has an insignificantly lower specific activity compared to PTRec 74 (1147 U/mg vs. 1209 U/mg).

In summary, two chimera with improved thermal stability at 90° C. and 95° C., without loss of specific activity, were identified.

Another added value is the low sequence identity (<70%) of the chimera to the E. coli phytase (PTRec 74: 66.8% and PTRec 77: 64% compared to E. coli WT) and to described commercial phytases (Hafnia alvei 81.8%, Citrobacter braakii 68.6%, Yersinia Mollaretii 85.2%, Hafnia compared to Yersinia 69.2%,), as they are chimaeras recombined from different enzymes.

1. A phytase chimera having an amino acid sequence according to SEQ ID NO:1 or SEQ ID NO:2, or an amino acid sequence having at least 82%, 84%, 86%, 88%, 90%, 91%, 92 ° /0, 93%, 94%, 95%, 96%, 97%, 98%, or 99 ° /0 sequence identity thereto.

2. The phytase chimera according to claim 1, wherein the amino acid sequence comprises a sequence portion having amino acids 92 to 415 of the sequence ac-cording to SEQ ID NO:1, or a sequence portion having at least 90%, 91%, 92%, 93%, 94%, 95%, 96%, 97%, 98%, or 99% sequence identity thereto.

3. The phytase chimera according to claim 1, having an amino acid sequence according to SEQ ID NO:1, SEQ ID NO:2, SEQ ID NO:8, SEQ ID NO:9, SEQ ID NO:10 or SEQ ID NO:11, or an amino acid sequence having at least 90%, 91%, 92%, 93%, 94%, 95%, 96%, 97%, 98%, 99% sequence identity thereto.

4. The phytase chimera according to claim 1, wherein the amino acid sequence has at least one substitution compared to the sequence according to SEQ ID NO:1 or SEQ ID NO:2.

5. The phytase chimera according to claim 1, having
a) a phytase stability characterized by a T5015 value, a T5030 value or a T5060 value of at least 50° C., preferably at least 60° C., more preferably at least 70° C., more preferably at least 80° C., most preferably at least 90° C., wherein the T5015 value, the T5030 value and the T5060 value are defined as the temperatures at which a 50 μg/mL preparation of the phytase chimera still has 50% residual activity after 15, 30 or 60 minutes of exposure, respectively as compared to a correspondingly long exposure at 25° C.; and/or
b) a specific phytase activity of at least 500 U/mg, preferably at least 700 U/mg, most preferably at least 900 U/mg,
if the residual activity and/or the phytase activity is determined on the basis of a reaction of 0.4 mM IP6 in 50 mM NaOAc, pH 4.9, at 60° C. over a period of 6 min.

6. A nucleic acid having a nucleic acid sequence encoding a phytase chimera according to claim 1.

7. A vector comprising a nucleic acid according to claim 6.

8. A host cell with a nucleic acid according to claim 6.

9. A process for producing a phytase chimera according to claim 1, comprising:
culturing a host cell with a nucleic acid having a nucleic acid sequence encoding the phytase chimera under conditions to cause expression of the nucleic acid sequence encoding the phytase chimera, and recovering the phytase chimera.

10. Use of a phytase chimera according to claim 1 in food and animal feed production.

11. A host cell with a vector according to claim 7.

12. The process of claim 9, wherein the host cell has a vector comprising the nucleic acid.
